(19) United States
(12) Patent Application Publication
(10) Pub. No.: US 2011/0060332 A1
(43) Pub. Date: Mar. 10, 2011

(54) Electrocautery device with combination of suction and light emission

(75) Inventor: Ming-Huei Cheng, Taipei City (TW)

(73) Assignees: Ming-Huei Cheng, Taipei City (TW); Steve L. Henry, Taipei City (TW); Holger Engel, Taipei City (TW)

(21) Appl. No.: 12/801,295

(22) Filed: Jun. 2, 2010

(30) Foreign Application Priority Data
Sep. 9, 2009 (TW) 098130354

(51) Int. Cl. A61B 18/14

(52) U.S. Cl. 606/42; 606/49

(57) ABSTRACT

An electrocautery device is disclosed, which includes: a shell body having a housing and an internal partition connecting to the housing to define a receiving space and at least one air-removal channel, where the housing has at least one first opening, at least one second opening and at least one third opening at a first end, and has at least one fourth opening at an opposite second end, the first and second openings connect to the receiving space, and the third and fourth openings connect to the air-removal channel; at least one electrode-blade penetrating through the first opening into the receiving space; at least one light emitting component arranged at the first end; and a circuit component electrically connecting the electrode-blade and the light emitting component and provided for electrical connection of an external power supply. The electrocautery device has both abilities of illumination and exhausting harmful gases.

### BACKGROUND OF THE INVENTION

1. Field of the Invention

The present invention relates to an electrocautery device and, more particularly, to an electrocautery device capable of illumination and readily removing gases generated during operation and being suitable for use in surgery.

2. Description of Related Art

With the progress of science and technology, medical devices and methods have been continuously developed. In modern surgery, electrocautery devices are important and commonly applied in various processes. For example, an electrocautery device can be used for cauterizing a wound, or be used together with an endoscope on an abdominal organ to stop bleeding or to excise an abdominal tumor. Accordingly, by using an electrocautery device, conventional laparotomy for excising partial tissue of the abdominal cavity can be replaced. In addition, in the case of using an electrocautery device together with an endoscope in abdominal surgery, the surgery wound is smaller in comparison to conventional laparotomy, and thereby the danger can be reduced and recovery time can be shortened.

FIG. 1 shows a conventional electrocautery device, i.e. an electrosurgical pencil. The electrosurgical pencil includes: a rod-shaped shell body 2, an electrode-blade 3 and a switch component 6. As shown in FIG. 1, there is a first opening 211a at the first end 211 of the shell body 2, and the electrode-blade 3 penetrates the first opening 211a and connects to the shell body 2. The internal circuit of the electrosurgical pencil penetrates the shell body 2 and connects to an external power supply 8 at the second end 212 opposite to the first end 211. In addition, for switching between the cutting and coagulation functions of the electrosurgical pencil, a switch component 6 containing a cutting button 61 and a coagulation button 62 is disposed on the surface of the shell body 2. Accordingly, when the electrode-blade 3 is provided with an electrical current from a wire or radio frequency by the external power supply 8 and contacts a human body, the difficulty of current passing through the tissue of the human body with high resistance will cause high heat to be generated so as to destroy tissue and thereby to excise tissue or stop bleeding.

Since the internal construction of the human body is complex, sufficient light is necessary during cutting and coagulation operations so as to avoid insufficient light causing the doctor to accidentally injure other parts of the patient's body. In general, the lamps of the operation room are insufficient, and thereby a doctor wears a headlamp to enhance the visibility of the treated site. However, since the doctor's head will move during surgery, the corresponding movement of the lamplight causes inconvenience and thereby the surgery danger is aggravated.

Besides, since human tissue mostly consists of protein and carbohydrate, odors as well as carcinogens will be generated and fill the air through high heat necessary for cauterizing. The generated smoke may be irritative and thereby badly affect the surgery operation in addition to obstructing the treated site.

Accordingly, it is necessary to develop an electrocautery device capable of illuminating the treated site and readily removing harmful gases to favor the surgery performance and reduce danger.

### SUMMARY OF THE INVENTION

To achieve the object, the present invention provides an electrocautery device, including a shell body having a housing and an internal partition, in which the internal partition connects to the housing to define a receiving space and at least one air-removal channel, a first end of the housing has at least one first opening, at least one second opening and at least one third opening, a second end of the housing, opposite to the first end, has at least one fourth opening, the first opening and the second opening connect to the receiving space, and the third opening and the fourth opening connect to the air-removal channel so as to allow an external air-removal apparatus to connect to the air-removal channel through the fourth opening; at least one electrode-blade, penetrating through the first opening and disposed in the receiving space; at least one light emitting component, disposed at the first end and emitting light through the second opening; and a circuit component, electrically connecting to the electrode-blade and the light emitting component and provided for connecting to an external power supply.

In the present invention, the light emitting component is integrated into the electrocautery device such that the light emitting component can be moved together with the electrode-blade to illuminate treated sites. Accordingly, the electrocautery device according to the present embodiment can avoid the problem of insufficient illumination and inappropriate movement of light from the headlamp, occurring in an external built-in lighting device. Through the electrocautery device, a doctor can accurately perform surgery for incision, excision and cauterizing on treated sites and the problem of insufficient illumination aggravating risks can be avoided. Additionally, in the present invention, an air-removal channel is integrated into the electrocautery device such that the gases (such as carcinogens or steam) generated during a cutting or cauterizing operation by the electrode-blade can be immediately extracted. Accordingly, during surgery, a doctor can be protected from carcinogens and irritant substances and the problem of steam obstructing the doctor's view can be avoided.

In the electrocautery device according to the present invention, the internal partition can divide the internal space within the housing into a receiving space and at least one air-removal channel separate from each other. Herein, the receiving space and the air-removal channel may be axially arranged in the shell body, in which the receiving space is provided for placing circuits and components and the air-removal channel is used for connecting to the external air-removal apparatus so as to provide air-removal function to the electrocautery device. In the present invention, the air-removal channel and the light emitting component are not limited in number. If necessary, more air-removal channels and light emitting components can be designed in the electrocautery device to enhance the efficiency in removing gases and providing illumination.

In the above-mentioned electrocautery device, the first end of the shell body may be rod shaped, and the electrode-blade may be separable from the shell body. In addition, according to the requirement, the electrocautery device may be unipolar typed or bipolar typed. As a bipolar electrocautery device, the first end of the shell body may be structured in a clamping form, which has two clamping arms. Herein, the first openings are individually located at the terminals of the clamping arms for placing the electrode-blades, such that the terminals of the clamping arms can individually connect to the electrode-blades to form a bipolar electrocautery device. Moreover, both the second opening and the third opening may be located at the terminal of the same clamping arm. Alternatively, the second opening is located at the terminal of one clamping arm, and the third opening is located at the terminal of the other clamping arm.

In the electrocautery device, according to the requirement, the circuit component may be disposed in the receiving space or be disposed outside of the shell body. The circuit component may electrically connect to a switch component so as to turn on/off the light emitting component and the electrode-blade. The switch component may be disposed on the surface of the housing, for example, as a hand-controlled switch, so as to allow an operator to manually switch the functions of the electrocautery device directly. Alternatively, through the circuitry, the switch component may be disposed outside of the shell body, for example, as a foot-controlled switch, so as to allow an operator to switch the functions of the electrocautery device directly by foot. Herein, the switch component may have plural buttons for individually turning on/off the light emitting component and actuating the cutting and coagulation functions of the electrode-blade.

Besides, the light emitting component of the electrocautery device may be disposed at the housing of the shell body or may penetrate the second opening and be disposed in the receiving space near the first end. In the case of placing the light emitting component in the receiving space near the first end, a light guide pipe may be selectively disposed at the second opening to guide and concentrate the light from the light emitting component. Herein, the light guide pipe may be flexible and capable of changing the pathway of light from the light emitting component. The type of the light emitting component is not limited and preferably is a light emitting diode, such as blue light emitting diode, white light emitting diode, yellow light emitting diode and so on.

The electrocautery device according to the present invention may further include an air-removal guide pipe disposed at the third opening of the first end and connecting to the air-removal channel. Accordingly, the air-removal inlet will be closer to the electrode-blade, and thereby the greater part of gases generated during cauterizing the treated site by the electrocautery device can be immediately removed. The air-removal guide pipe may be flexible, such that the orientation and length of the air-removal guide pipe can be adjusted according to the flow direction of gases from the treated site to minimize the harmful effect of the gases. In addition, the caliber of the air-removal guide pipe may get greater and greater as it approaches closer and closer the terminal of the electrode-blade. The terminal of the air-removal guide pipe may be canted such that its canted terminal can face the treated site during operation to thoroughly remove the generated gases.

In the above-mentioned electrocautery device, the relative locations of the first opening, the second opening and the third opening are not limited. For example, the first opening may be located between the second opening and the third opening. Additionally, the second opening and the third opening are not limited in number and can be determined according to the number of the light emitting components and the air-removal channels.

Other objects, advantages, and novel features of the invention will become more apparent from the following detailed description when taken in conjunction with the accompanying drawings.

### BRIEF DESCRIPTION OF THE DRAWINGS

FIG. 1 shows a conventional unipolar electrocautery device.

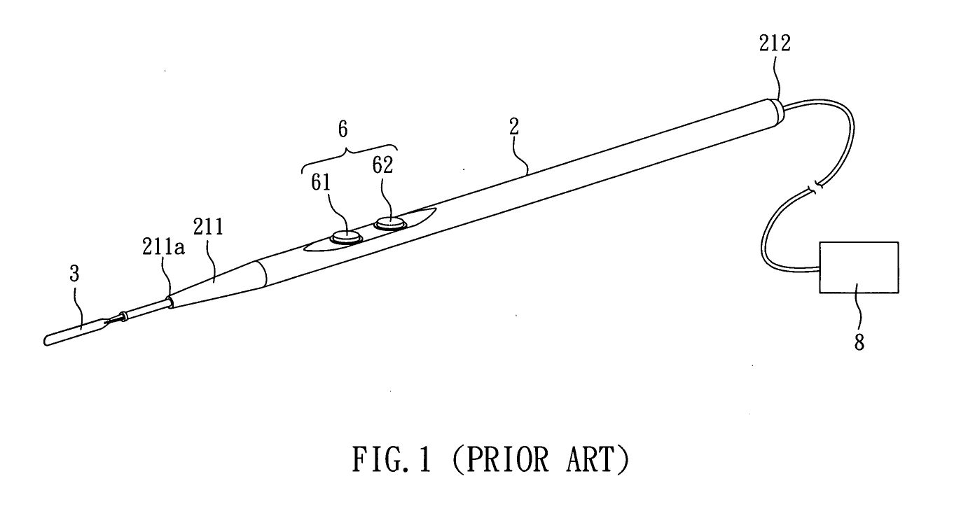

FIG. 2 shows a cross-sectional view of a unipolar electrocautery device according to Example 1 of the present invention.

FIG. 3 shows a cross-sectional view of a separable unipolar electrocautery device according to Example 2 of the present invention.

FIGS. 4A to 4C show the arrangement of the light emitting component(s) and the air-removal channel(s) at the first end of the housing of the shell body according to various aspects in Example 3 of the present invention.

FIGS. 5A to 5C show the structure of the first end according to the bipolar electrocautery device in Example 4 of the present invention.

### DETAILED DESCRIPTION OF THE PREFERRED EMBODIMENT

### Example 1

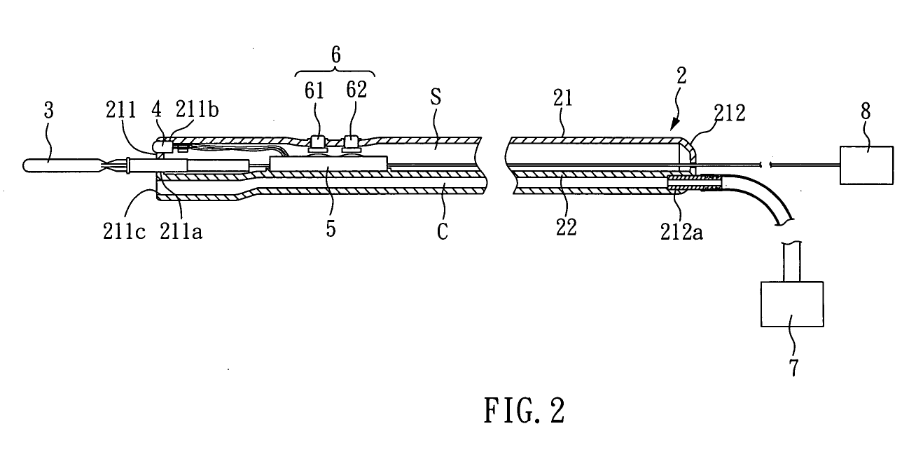

FIG. 2 shows a cross-sectional view of a unipolar electrocautery device according to the present invention. As shown in FIG. 2, the electrocautery device according to the present invention includes: a shell body 2, an electrode-blade 3, a light emitting component 4, a circuit component 5 and a switch component 6.

The above-mentioned shell body 2 is hollow and has a housing 21 and an internal partition 22 connecting to the housing 21. Through the housing 21 and the internal partition 22, the internal space of the shell body 2 is defined into a receiving space S and an air-removal channel C. The housing 21 has a first end 211 and a second end 212 opposite to the first end 211.

At the first end 211 of the housing 21, there are a first opening 211a, a second opening 211b and a third opening 211c. Additionally, there is a fourth opening 212a at the second end 212. The first opening 211a and the second opening 211b connect to the receiving space S, and the third opening 211c and the fourth opening 212a connect to the air-removal channel C.

An external air-removal apparatus 7 can connect to the air-removal channel C through the fourth opening 212a at the second end 212 of the housing 21 so as to provide an air-removal function to the electrocautery device. If the receiving space S and the air-removal channel C are isolated and cannot communicate with each other, the electrocautery device will have better efficiency for air removal. Thereby, it can be known that the receiving space S is provided for placing circuits and components, and the air-removal channel C is used for connecting to the external air-removal apparatus 7 so as to provide an air-removal function to the electrocautery device. The arrangement of the receiving space S and the air-removal channel C is not specifically limited. As shown in FIG. 2, they may be axially arranged in the shell body 2.

The electrode-blade 3 is a unipolar blade, and the head end of the blade may be flat and sharp (determined according to the requirement). Its connecting end penetrates through the first opening 211a and is disposed in the receiving space S to connect to the shell body 2. The light emitting component 4 is disposed in the second opening 211b at the first end 211 of the housing 21. That is, the light emitting component 4 is directly disposed on the housing 21 of the shell body 2.

The circuit component 5 is disposed in the receiving space S and its circuit penetrates through the shell body 2 to electrically connect to an external power supply 8. Also, the circuit component 5 electrically connects to the electrode-blade 3 and the light emitting component 4 by means of its circuit. Accordingly, due to the circuit component 5, the external power supply 8 can supply electrical power to the electrode-blade 3 and the light emitting component 4 for operation thereof.

The switch component 6 is disposed on the surface of the housing 21 of the shell body 2 and extends through the housing 21 to electrically connect to the circuit component 5. The switch component 6 contains a cutting button 61 and a coagulation button 62 so as to respectively actuate either the cutting function or the coagulation function of the electrode-blade 3. The switch component 6 may further contain a button for actuating the light emitting component 4 in addition to various other function buttons. Thereby, the switch component 6 in the present embodiment is hand controlled so as to allow a doctor to manually switch between functions of the electrocautery device.

In the present embodiment, the light emitting component 4 is integrated into the electrocautery device, and thereby the light emitting component 4 can be moved together with the electrode-blade to illuminate treated sites. Accordingly, the electrocautery device according to the present embodiment can avoid the problems of insufficient illumination and light misdirection due to head movement, occurring in an external built-in lighting device. Through the electrocautery device, a doctor can accurately perform surgery on treated sites to ensure successful surgery. Additionally, since the electrocautery device contains the air-removal channel C that can connect to an external air-removal apparatus 7, the external air-removal apparatus 7 can readily remove gases generated during the cutting and cauterizing operations by means of the third opening 211c and the air-removal channel C. Thereby, even if carcinogens or fog are generated during surgery, the doctor can be protected from the risks of exposure thereto.

### Example 2

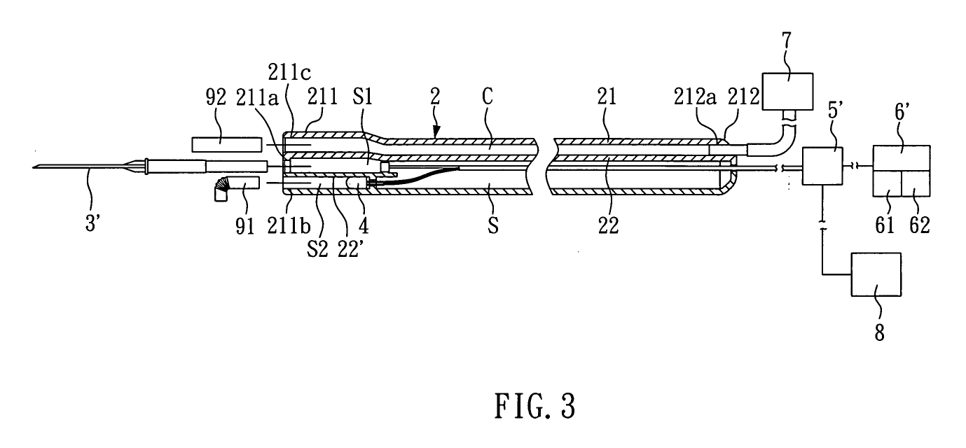

FIG. 3 shows a cross-sectional view of another unipolar electrocautery device according to the present invention. As shown in FIG. 3, the electrocautery device according to the present invention includes: a shell body 2, an electrode-blade 3′, a light emitting component 4, a circuit component 5, a switch component 6, a light guide pipe 91 and an air-removal guide pipe 92.

The structure of the shell body 2 according to the present embodiment is the same as that illustrated in Example 1, except that the shell body 2 according to the present embodiment further has a sub-internal partition 22′. The sub-internal partition 22′ connects to the housing 21 to define the partial space of the receiving space S as a light-disposed space S2 to place the light emitting component 4 therein. In addition, the sub-internal partition 22′ and the internal partition 22 define the partial space of the receiving space S as a blade-disposed space S1 for placing the electrode-blade 3′ therein.

The electrode-blade 3′ is a unipolar blade that can be separated from the shell body 2. Its head end is thin and sharp, and its connecting end penetrates through the first opening 221a and is disposed in the receiving space S. The light emitting component 4 is a light emitting diode and disposed in the light-disposed space S2. The light from the light emitting component 4 can pass through the light-disposed space S2 and the second opening 211b and then illuminate the treatment sites of a patient.

The light guide pipe 91 is flexible and can be used to change the pathway of light from the light emitting component 4. The light guide pipe 91 penetrates through the second opening 211b and is placed in the light-disposed space S2. Accordingly, the light guide pipe 91 can guide the light from the light emitting component 4 and focus the light on the treatment sites.

The air-removal guide pipe 92 is flexible and placed in the third opening 211c at the first end 211 to connect with the air-removal channel C. Accordingly, the air-removal inlet at the third opening 211c is closer to the electrode-blade 3′, and thereby gases generated during operation of the electrocautery device can be removed immediately. Additionally, the suction can be controlled by the external air-removal device 7 to minimize the harmful effects of the gases.

The circuit component 5′ is disposed outside of the shell body 2 and its circuit penetrates through the housing 21 of the shell body 2 and reaches the receiving space S and electrically connects to the light emitting component 4 and the electrode-blade 3′. The circuit component 5′ outside of the shell body 2 electrically connects to an external power supply 8, and the external power supply 8 can supply electrical power to the electrode-blade 3′ and the light emitting component 4 by means of the circuit component 5′.

The switch component 6′ is also disposed outside of the shell body 2 and electrically connects to the circuit component 5′ outside of the shell body 2. If necessary, the switch component 6′ and the circuit component 5′ may be integrated with each other, and electrically connects to the external power supply 8, the light emitting component 6 and the electrode-blade 3′ so as to switch between the cutting function and coagulation function of the electrode-blade 3′ and turn on the light emitting component 4. Accordingly, the switch component 6′ in the present embodiment can be foot controlled, such that a doctor can accordingly switch between the functions of the electrocautery device.

### Example 3

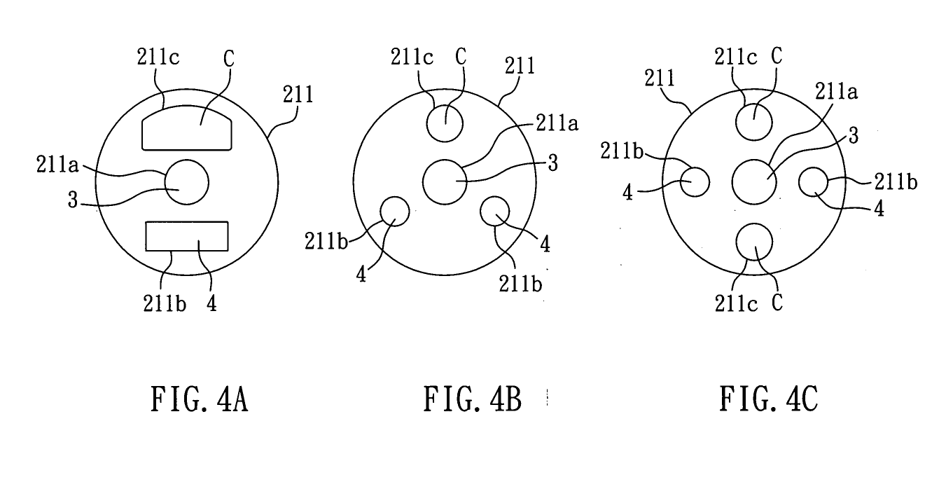

FIGS. 4A to 4C show the arrangement of the light emitting component(s) 4 and the air-removal channel(s) C at the first end 211 of the housing 21 of the shell body 2.

The first aspect is shown in FIG. 4A. The first opening 211a, through which the electrode-blade 3/3′ penetrates to be disposed in the receiving space S, is disposed at the center of the housing 211 of the shell body 2 and between the second opening 211b (i.e. the outlet for light from the light emitting component 4) and the third opening 211c (i.e. the outlet of the air-removal channel C).

FIG. 4B shows the second aspect. For placing more light emitting components 4, another second opening 211b is disposed at the first end 211 of the housing 21 of the shell body 2. Accordingly, the increase in the number of the light emitting components 4 results in enhancing the illumination of the treatment sites.

As for the third aspect, FIG. 4C shows that another third opening 211c is disposed at the first end 211 of the housing 21 of the shell body 2 and a further air-removal channel C is correspondingly defined by the internal partition 22 of the shell body 2. Since fog and gases generated during operation of the electrocautery device can be more widely removed by increasing air-removal openings, the efficiency for removing gases is enhanced.

In the electrocautery device according to the present invention, the relative locations and numbers of the first openings 211a, the second openings 211b and the third openings 211c are not limited to the above-mentioned case, and can be modified according to the actual condition.

### Example 4

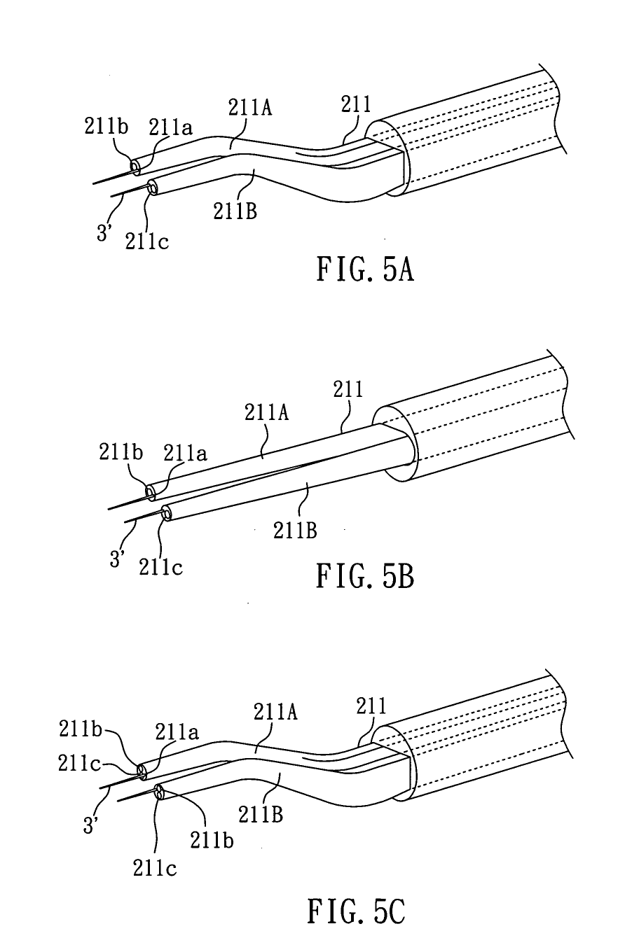

FIGS. 5A to 5C show the structure of the first end 211 of the shell body 2 according to the bipolar electrocautery device of the present invention. Herein, the other parts except the first end 211 are structured as the above-mentioned example.

As shown in FIG. 5A, the first end 211 of the shell body 2 is structured in a clamping form, which has two clamping arms, 211A and 211B. Herein, the two clamping arms can be equal to each other in length, and be slightly curved. The first openings 211a are individually located at the terminals of the clamping arms 211A and 211B, such that the electrode-blades 3′ can penetrate the first openings 211a and be fastened in the clamping arms 211A and 211B. In addition, the second opening 211b is located at the clamping arm 211A to allow the light emitting component 4 (not shown in the figure) to emit light from the second opening 211b to the treated site. Furthermore, the third opening 211c is located at the terminal of the clamping arm 211B, such that exhaust gases can be removed through the third opening 211c. Accordingly, during using the bipolar electrocautery device of the present example, the clamping arm 211A can illuminate the treated site and the clamping arm 211B can remove exhaust gases generated during cauterizing.

FIG. 5B shows another aspect, in which the clamping arms 211A and 211B are straight rather than curved, unlike the structure shown in FIG. 5A.

FIG. 5C shows further another aspect, in which each of the clamping arms 211A and 211B has both the second opening 211b and the third opening 211c at its terminal, unlike the structure shown in FIG. 5A.

Although the present invention has been explained in relation to its preferred embodiment, it is to be understood that many other possible modifications and variations can be made without departing from the scope of the invention as hereinafter claimed.

What is claimed is:

1. An electrocautery device, comprising:
a shell body having a housing and an internal partition, wherein the internal partition connects to the housing to define a receiving space and at least one air-removal channel, a first end of the housing has at least one first opening, at least one second opening and at least one third opening, a second end of the housing, opposite to the first end, has at least one fourth opening, the first opening and the second opening connect to the receiving space, and the third opening and the fourth opening connect to the air-removal channel so as to allow an external air-removal apparatus to connect to the air-removal channel through the fourth opening;
at least one electrode-blade, penetrating through the first opening and disposed in the receiving space of the shell body;
at least one light emitting component, disposed at the first end and emitting light through the second opening; and
a circuit component, electrically connecting to the electrode-blade and the light emitting component and provided for connecting to an external power supply.

2. The electrocautery device as claimed in claim 1, wherein the first end of the shell body is structured in a rod shape.

3. The electrocautery device as claimed in claim 2, wherein the electrode-blade is separable from the shell body.

4. The electrocautery device as claimed in claim 1, wherein the first end of the shell body is structured in a clamping shape and has two clamping arms, and the first opening is located at each terminal of the clamping arms.

5. The electrocautery device as claimed in claim 4, wherein both the second opening and the third opening are located at the terminal of at least one of the clamping arms.

6. The electrocautery device as claimed in claim 4, wherein the second opening is located at the terminal of one of the clamping arms, and the third opening is located at the terminal of the other of the clamping arms.

7. The electrocautery device as claimed in claim 1, wherein the circuit component is disposed in the receiving space.

8. The electrocautery device as claimed in claim 1, further comprising a switch component electrically connecting to the circuit component.

9. The electrocautery device as claimed in claim 8, wherein the switch component is disposed on the surface of the housing.

10. The electrocautery device as claimed in claim 1, wherein the light emitting component is disposed in the receiving space at the first end.

11. The electrocautery device as claimed in claim 10, further comprising a light guide pipe disposed at the second opening of the first end.

12. The electrocautery device as claimed in claim 11, wherein the light guide pipe is flexible.

13. The electrocautery device as claimed in claim 1, further comprising an air-removal guide pipe disposed at the third opening of the first end and connecting to the air-removal channel.

14. The electrocautery device as claimed in claim 13, wherein the air-removal guide pipe is flexible.

15. The electrocautery device as claimed in claim 1, wherein the first opening is located between the second opening and the third opening.

16. The electrocautery device as claimed in claim 1, wherein the light emitting component is a light emitting diode.
